(19) United States
(12) Patent Application Publication
(10) Pub. No.: US 2010/0093014 A1
(43) Pub. Date: Apr. 15, 2010

(54) SCREENING FOR BONE ANABOLIC FACTORS

(75) Inventors: Anita Vibsig Neutzskywulff, Frederiksvaerk (DK); Kim Henriksen, Hillerod (DK); Morten Karsdal, Copenhagen (DK)

Correspondence Address: EDWARDS ANGELL PALMER & DODGE LLP, P.O. BOX 55874, BOSTON, MA, 02205, US

(73) Assignee: NORDIC BIOSCIENCE IMAGING A/S, Herlev (DK)

(21) Appl. No.: 12/450,027

(22) PCT Filed: Apr. 14, 2008
(86) PCT No.: PCT/EP2008/054515

§ 371 Date: Sep. 8, 2009

(30) Foreign Application Priority Data
Apr. 16, 2007 (GB) 0707301.8

(51) Int. Cl. C12Q 1/02

(52) U.S. Cl. 435/29

(57) ABSTRACT

A method for screening for modulators of the secretion by an osteoclast of a wnt or a wnt signal enhancer comprises exposing osteoclasts in culture to a compound to be screened, exposing a wnt sensitive detection system to conditioned medium from said osteoclast culture, and determining whether a wnt signal is present in said medium by assaying for wnt mediated activation of bone formation by osteoblasts or by wnt mediated activation of LRP5 and or LPR6 signalling in a cell by detection of β catenin or detection of translocation of disheveled, axin, or Frat1 to the cell membrane of said cell.

In normal healthy individuals bone formation is coupled to bone resorption in a tight equilibrium. When this delicate balance is disturbed, the net result is a pathological situation, such as osteopetrosis or osteoporosis. Human osteopetrosis, caused by mutations of proteins involved in the acidification of the resorption lacuna (ClC-7 or the a3-V-ATPase), is characterized by decreased resorption in face of normal or even increased bone formation. Mouse mutations leading to ablation of osteoclasts, e.g. loss of M-CSF or c-fos lead to secondary negative effects on bone formation, in contrast to mutations where bone resorption is abrogated with sustained osteoclast numbers, such as the c-src mice. These data indicate a central role for osteoclasts, and not necessarily their resorptive activity, in the control of bone formation.

Bone is a dynamic tissue, which is continuously remodeled throughout life not only to maintain calcium homeostasis but also to repair micro-damage, and thus maintain bone quality. This continuous remodeling of bone involves the function of cells that strive to achieve a coordinated and balanced resorption of old bone (osteoclasts) and of those responsible for adequate formation of new bone (osteoblasts), in a local, coordinated and sequential manner referred to as coupling.

The coupling process is understood as a bone formation response that results from bone resorption, with an amount of bone formed equal to that resorbed. Uncoupling occurs when the balance between formation and resorption is dissociated, which can lead to either osteopetrosis or osteoporosis. Although it has long been appreciated that bone formation is tightly coupled to bone resorption in normal adult bone turnover, this coupling can be dissociated in some circumstances, for example during skeletal growth, in postmenopausal osteoporosis and in some, but not all, osteopetrotic mutations.

Loss of ovarian sex steroids in postmenopausal women results in an acceleration of bone turnover with predominance of bone resorption over bone formation. The related negative calcium balance promotes bone loss, increases bone fragility, and thereby the risk of future fractures. A rational approach to counter these unwanted processes is to inhibit bone resorption, which until now also has led to inhibition of bone formation, due to the coupling between these cellular events.

Osteoclast activity has traditionally been viewed as restricted to bone resorption. It has now been established that it is not restricted to this (7,8) and that there is a further activity of osteoclasts, namely stimulation of bone formation. We demonstrate that osteoclasts secrete an anabolic factor which stimulates bone formation by osteoblasts and that this factor is a wnt. Based on this, the invention now provides a method for screening for modulators of the secretion by an osteoclast of a wnt or a wnt signal enhancer, which method comprises exposing osteoclasts in culture to a compound to be screened, exposing a wnt sensitive detection system to conditioned medium from said osteoclast culture, and determining whether a wnt signal is present in said medium.

Preferably, the level of the wnt signal is detected quantitatively. Compounds providing an enhancement of the wnt signal in the conditioned medium will be candidates for further investigation as bone anabolic agents or lead compounds.

Where a wnt is detected, it may preferably be an anabolic osteoclast-to-osteoblast signal.

Preferred screening assays will be detect both an anabolic effect on osteoblasts and the wnt related nature of the secreted signal. More than one screening assay as described herein may be performed on each compound.

Wnt signal enhancers will include osteoclast secreted inhibitors of factors that in turn inhibit wnt signals or any other factor which increases the effect on a wnt signalling pathway of a wnt signal. The effect of such an enhancer in the screening protocols described herein will be expected to resemble an increased secretion of a wnt itself.

A said wnt signal is optionally detected in said conditioned medium by assaying for wnt mediated activation of bone formation by osteoblasts. This may be as described in Examples 2 and 3.

Alternatively or additionally, said wnt signal is detected in said conditioned medium by assaying for wnt mediated activation of LRP5 and or LRP6 signalling in a cell. Said LRP5 and or LRP6 signalling may be detected by detection of β-catenin. This may be by detection of translocation of β-catenin to the nucleus of said cell. Other wnt signal detection methods are described below also.

Wnts are a known family of signalling proteins in which over a dozen distinct wnts have been identified. They act in a complex signalling cascade via binding to a co-receptor complex including G Protein coupled receptors and receptors known as LRP5 and/or LRP6 to produce diverse effects. These include renewal of stem cells, stimulation of pre-osteoblast replication, induction of osteoblastogenesis, and inhibition of osteoblast and osteocyte apoptosis. It is known that osteoblasts are stimulated to form bone by wnts, but the nature of a natural source of a wnt signal to osteoblasts has not previously been established.

By screening for compounds which will stimulate osteoclasts to secrete their bone anabolic wnt signal or which will enhance the effect of the secreted signal, the way is opened for the development of bone anabolic drugs and the decoupling of bone resorption from bone formation. Such drugs will be useful in treating diseases in which bone resorption is out of balance and is excessive with respect to the rate of bone formation by osteoblasts. Such diseases include osteoporosis (including post-menopausal osteoporosis, sex hormone deficiency osteoporosis, osteoporosis as a symptom or side effect of ulcerative colitis or Crohn's disease or its treatment by corticosteroids, senile osteoporosis, drug induced osteoporosis, disuse osteoporosis), hyperparathyroidism, Paget's disease, hypercalcemia of malignancy, osteolytic lesions produced by bone metastasis, bone loss due to immobilisation, bone loss due to spinal fracture, and osteomalacia.

To screen compounds for activation of secretion or the enhancement of the efficiency of signalling of the wnts, one may use assays based on wnt mediated activated of LRP5 mediated signalling. LRP5 mediates signals through β-catenin, which upon activation of LRP5 by wnts will translocate to the nucleus of the cells (1). Suitably, one may use a cell line such as the mouse cell line MC3T3E1, or another cell line or primary transfected cells which endogenously contain all the LRP5/wnt signalling components. MC3T3E1 cells are known to show translocation of β-catenin in response to wnts. The translocation may be screened using immunofluorescence or immunoperoxidase detection of β-catenin in the presence or absence of conditioned media from osteoclasts treated with various compounds to augment wnt production.

Alternatively, wnt signal present in osteoclast conditioned medium can be detected in a screening method by exposure of cells (stably or transiently) transfected with a reporter system containing TCF/LEF response elements in the promoter region of a reporter gene, such as a luciferase gene or other gene coding for a detectable enzyme. An example of such a reporter system is the TOPflash luciferase reporter system.

Activation of the LRP5 pathway leads to translocation of disheveled (dsh), Frat1 and Axin from the cytosol to the cell surface (5), and therefore an alternative screening procedure is to assay for these components being present in response to conditioned media from osteoclasts using relocalisation assays as described for β-catenin.

Furthermore, the recruitment of transcription factors of the TCF/LEF family to the nucleus can also be assayed using a translocation assay (6). This may be done using cells treated in the presence of conditioned media or the corresponding controls for a period such as 24 hours, and then fixed for immunolocalisation using antibodies specific for the transcription factors, e.g. TCF1. The procedure may be similar to that described in Example 5 below. Briefly, this procedure is as follows: Incubation with a TCF1 specific antibody, and the detection of the antibody using a labelled secondary antibody, which will allow following translocation to the nucleus. The cells used should be competent for the required signalling pathway and may be osteoblasts, or an osteoblastic cell line, and may for instance be MC3T3-E1 cells.

The invention will be further illustrated and described in the following Examples, in which reference is made to the attached drawings, the content of which may briefly be described as follows:

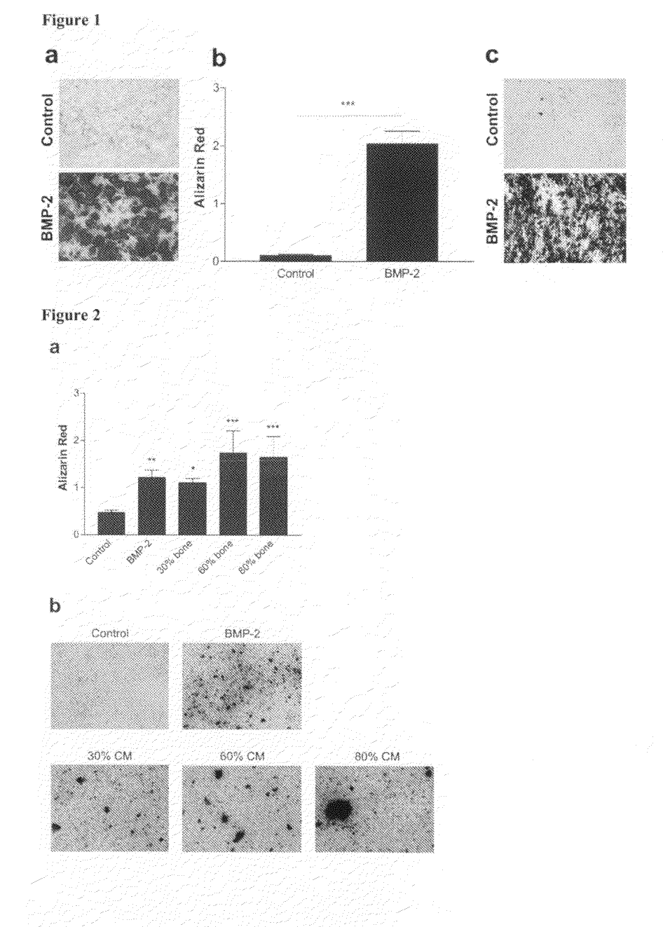

FIG. 1, panels a, b and c, shows results obtained in Example 1;

FIG. 2, panels a and b, shows results obtained in Example 2;

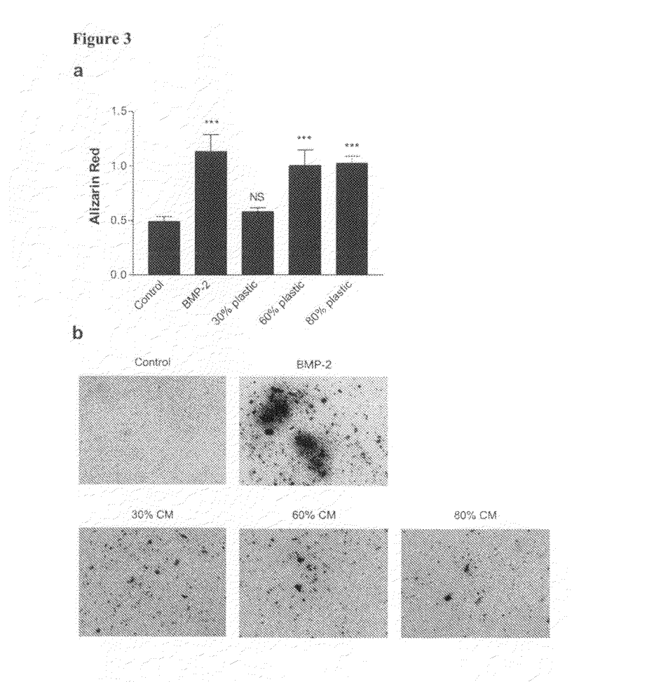

FIG. 3, panels a and b, shows results obtained in Example 3; and

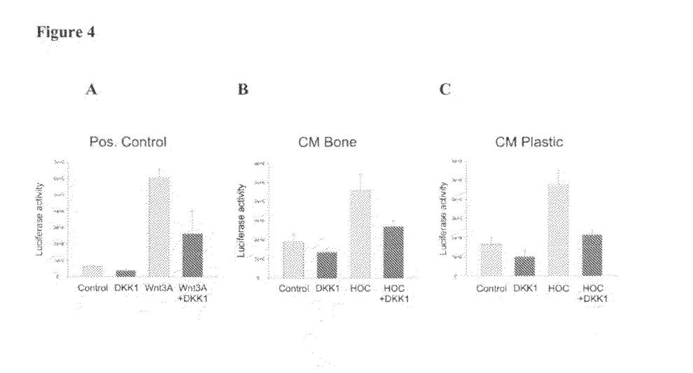

FIG. 4, panels A, B and C, shows results obtained in Example 4.

### EXAMPLES

### Example 1

### MC3T3-E1 Cells Form Nodules in the Presence of Anabolic Stimuli

We investigated whether the MC-3T3-E1 pre-osteoblast cell line (2)] responded to anabolic stimuli, and therefore was suitable for investigation of signals potentially present in the conditioned medium from osteoclasts. 3T3-E1 cells were cultured in the presence or absence of 10 ng/mL of Bone Morphogenetic Protein 2 (BMP-2), which is known to induce bone formation.

The pre-osteoblast murine MC-3T3-E1 cells were cultured in αMEM containing 10% of heat-inactivated FBS with a change of medium every 2^{nd} or 3^{rd} day and then lifted by trypsin treatment when they reached confluence, followed by reseeding of a suitable ratio of the cells (1:3-1:5). To induce bone nodule formation the MC3T3-E1 cells were lifted by trypsin and reseeded in 24-well plates at a density of 75,000 cells/24-well, and then cultured in the presence of 50 μg/mL ascorbic acid and 10 mM β-glycerol-phosphate, for three weeks, with a medium change every 2^{nd} or 3^{rd} day.

Nodule formation was measured using Alizarin Red. After three weeks of culture the osteoblasts cultures were stopped by fixation in 3.7% formaldehyde in PBS for 20 minutes. The cells were stained in 40 mM Alizarin Red pH 4.2 for 10 minutes, and washed thoroughly. Digital histographs of representative areas of stained nodules were taken using an Olympus IX-70 light microscope equipped with an Olympus C5050 Zoom digital camera. Subsequently, the dye was extracted from the cells by incubation in 10% cetylpyridinium chloride for 15 minutes while shaking in the dark, and finally the absorbance at 561 nm was measured using an ELISA reader.

Nodule formation was also measured by Von Kossa staining of the bone nodules. After stopping of the culture as above, the bone nodules were stained in 2.5% AgNO₃ for 30 minutes under intense illumination. The cells were washed thoroughly in milliQ and the staining was further developed by incubation in 1% pyrogallol for 30 seconds. After washing in milliQ, the staining was fixed in 5% sodium thiosulphate for 5 minutes and washed again in milliQ. Finally, digital histographs of representative areas of stained nodules were taken using an Olympus IX-70 light microscope equipped with an Olympus C5050 Zoom digital camera.

As seen in FIG. 1a-c, BMP-2 significantly induced bone nodule formation by the MC-3T3-E1 cells as measured both using Alizarin Red (FIGS. 1a and b) and Von Kossa staining (FIG. 1c).

These results show that the MC-3T3-E1 cells can indeed be driven toward bone formation in the presence of anabolic stimuli.

### Example 2

### Resorbing Osteoclasts Secrete Anabolic Signals

To investigate whether normal human osteoclasts cultured on bone secreted bone anabolic signals, we collected CM from resorbing osteoclasts during a three-week period. To generate osteoclasts, human CD14+ monocytes were isolated using magnetic bead sorting as previously described [(3)] (4). Briefly, buffycoats diluted 1:1 in PBS were layered onto ficoll gradients and centrifuged, and the lymphocyte fraction was collected at the interphase. The lymphocytes were then washed and incubated with anti-CD14 beads (DYNAL Biotech) for 20 minutes, and finally isolated using a magnetic device. The monocytes were counted and seeded at a cell density of 150,000/cm² and cultured for 10 days in αMEM containing 10% of heat-inactivated fetal bovine serum (FBS) and 25 ng/mL M-CSF and 25 ng/mL RANKL (R&D Systems) with a medium change every 2^{nd} or 3^{rd} day to induce the formation of mature osteoclasts.

Mature human osteoclasts were lifted by trypsin digestion and subsequent scraping, and then reseeded on either bone slices at a density of 600,000 cells/well in 12-well plates. The osteoclasts were then cultured in medium containing RANKL, M-CSF and 10% heat-inactivated FBS for three weeks with a change of medium every second or third day. During each medium change the supernatants were collected and stored at −20° C. until further use. Before use in the osteoblast cultures the CM was filtered through a 0.45 μm low-protein binding filter to remove aggregates, cell fragments etc. from the solution.

The quality of the CM from the individual collection days was investigated by measurement of the resorption marker collagen type I fragment (CTX-I) using the CrossLaps for Culture kit (Nordic Bioscience Diagnostics), which was used according to the manufacturers instructions. The resorption data confirmed the presence of mature resorbing osteoclasts throughout the whole culture period (data not shown).

We then exposed MC-3T3-E1 cells to the CM in the indicated concentrations, and we found that CM from resorbing osteoclasts dose-dependently induced nodule formation as measured by Alizarin Red as above (FIG. 2a). The maximal level of induced bone nodule formation was achieved at 60%, reaching a level of 400% compared to the negative control. The induction of nodule formation was not quantitatively different from the induction observed using 10 ng/mL of BMP-2 as a positive control. We confirmed these findings by qualitative assessment of nodule formation using Von Kossa staining (FIG. 2b).

### Example 3

### Osteoclasts Secrete Non-Bone Derived Anabolic Signals

To clarify whether the signal derived from the bone matrix or from the osteoclasts themselves, we added CM from osteoclasts grown on plastic to osteoblasts, and investigated whether it was able to stimulate bone formation. First, we assured the high quality of the conditioned medium by measuring the osteoclast marker tartrate-resistant acid phosphatase (TRACP) activity using a colorimetric assay. The conditioned media were tested for TRACP activity by addition of 6 mM 4-nitrophenylphosphate and 25 mM sodium tartrate at pH5.5. The reaction products were quantified by measuring absorbance at 405 nm with 650 nm as reference using an ELISA reader.

The level of TRACP activity was high in the collected CM confirming that mature osteoclasts were present (data not shown).

Next, we evaluated whether the CM could induce nodule formation in the MC-3T3-E1 cells. As measured by the Alizarin Red method described above, CM dose-dependently induced bone formation with a maximal effect of 200%, which was reached at 60% of CM, and the effect was comparable to that observed with the positive control BMP-2 (FIG. 3a). In support of this, we qualitatively investigated the formation of bone nodules using Von Kossa staining, and as seen in FIG. 3b, we found that CM induced the formation of nodules to the same extent as BMP-2. Thus, our findings indicate that osteoclasts secrete non-bone derived signals inducing bone formation.

### Example 4

### Osteoclast Secrete Signals that Stimulate Wnt Signaling Specifically in Osteoblasts

Our recent data indicate that the activation of bone formation by the conditioned medium is caused by a wnt. Wnts are known osteoblast activators (1,5), but production of these by the osteoclasts has not been described.

We transiently infected UMR-106 cells (a rat sarcoma derived cell line) with a luciferase reporter system TOPflash containing 8 TCF/LEF response elements placed in the promoter region of the luciferase reporter gene. The TCF/LEF response elements specifically mediate activation by wnts, and as seen in FIG. 4A the positive control Wnt3A induces a strong activation. Furthermore, the Wnt inhibitor DKK1 inhibits this activation as expected.

To investigate the effect of secreted signals from osteoclasts we collected conditioned medium from osteoclasts cultured either on bone or on plastic, and compared their ability to induce activation of cells transiently transfected with the TCF/LEF reporter system to that of non-conditioned osteoclast culture medium. The cells were then treated for 24 hours with either control compounds, vehicle or the different conditioned media, after which the cells were lysed and the Luciferase activity was measured by a colorimetric assay.

Conditioned medium from the osteoclasts on bone specifically induced TCF/LEF activation, and this was inhibited by DKK1 (see FIG. 4B), confirming that the signal is a wnt. Furthermore, non-resorbing osteoclasts on plastic also secreted a signal leading to TCF/LEF activation, which again was inhibited by DKK1 (See FIG. 4C).

### Example 5

### The β-Catenin Translocation Assay

To perform a screening assay for modulation of the osteoclast production of wnt signal to osteoclasts, MC3T3-E1 cells were seeded on glass coverslips and allowed to attach. After attachment they were treated with various control compounds (wnt3A or LiCl positive controls), vehicle or conditioned media from osteoclasts for 24 hours. After treatment they were fixed in Lilly's fluid for 20 minutes, washed thoroughly in PBS, and then β-catenin was immunolocalized using a rabbit anti-β-catenin antibody from Cell Signalling Technology as a primary antibody, and Rabbit Envision from DakoCytomation as secondary antibody. Finally, the peroxidase reaction was visualized using DAB+ as a substrate and the nuclei of the cells were counterstained using Ehrlich's hematoxylin. The translocation was investigated using an Olympus BX-20 light microscope equipped with a 60× objective.

Treatment of the osteoclasts with compounds to be screened prior to the use of conditioned medium from the osteoclast culture in the above procedure provides the required screening assay.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference.

### REFERENCES

1. Martin, T. J., Quinn, J. M., Gillespie, M. T., Ng, K. W., Karsdal, M. A., and Sims, N. A. 2006. Mechanisms involved in skeletal anabolic therapies. Ann. NY Acad Sci. 1068:458-470.
2. Karsdal M. A, Andersen T A, Bonewald L, and Christiansen C. 2004. Matrix metalloproteinases (MMPs) safeguard osteoblasts from apoptosis during trans-differentiation into osteocytes: MT1-MMP maintains osteocyte viability. DNA & Cell Biology. 23:155-165.
3. Karsdal, M. A., Hjorth, P., Henriksen, K., Kirkegaard, T., Nielsen, K. L., Lou, H., Delaisse, J. M., and Foged, N. T. 2003. Transforming growth factor-beta controls human osteoclastogenesis through the p38 MAPK and regulation of RANK expression. J Biol Chem. 278:44975-44987.
4. Schaller, S., Henriksen, K., Sveigaard, C., Heegaard, A. M., Helix, N., Stahlhut, M., Ovejero, M. C., Johansen, J. V., Solberg, H., Andersen, T. L. et al. 2004. The chloride channel inhibitor n53736 prevents bone resorption in ovariectomized rats without changing bone formation. J Bone Miner Res. 19:1144-1153.
5. Krishnan, V., Bryant, H. U., and Macdougald, O. A. 2006. Regulation of bone mass by Wnt signaling. J Clin Invest. 116:1202-1209.
6. Glass & Karsenty Endocrinology, Mar. 29, 2007 Epub ahead of print
7. Karsdal M A, Martin T J, Bollerslev J, Christiansen C, Henriksen K. Are nonresorbing osteoclasts sources of bone anabolic activity? J Bone Miner Res. 2007 April; 22(4):487-94. PMID: 17227224 [PubMed—in process]
8. Martin T J and Sims N A. Osteoclast-derived activity in the coupling of bone formation to resorption. Trends Mol Med. 2005 February; 11(2):76-81

1. A method for screening for modulators of the secretion by an osteoclast of a wnt or a wnt signal enhancer, which method comprises exposing osteoclasts in culture to a compound to be screened, exposing a wnt sensitive detection system to conditioned medium from said osteoclast culture, and determining whether a wnt signal is present in said medium.

2. A method as claimed in claim 1, wherein said wnt is an anabolic osteoclast-to-osteoblast signal or said wnt signal enhancer is an enhancer of an anabolic osteoclast-to-osteoblast signal.

3. A method as claimed in claim 2, wherein a said wnt signal is detected in said conditioned medium by assaying for wnt mediated activation of bone formation by osteoblasts.

4. A method as claimed in claim 1, wherein a said wnt signal is detected in said conditioned medium by assaying for wnt mediated activation of LRP5 and or LRP6 signalling in a cell.

5. A method as claimed in claim 4, wherein said LRP5 and or LRP6 signalling is detected by detection of β-catenin.

6. A method as claimed in claim 5, wherein said signalling is detected by detection of translocation of β-catenin to the nucleus of said cell.

7. A method as claimed in claim 5, wherein said signalling is detected by detection of translocation of disheveled, axin and/or Frat1 to the cell membrane of said cell.

8. A method as claimed in claim 1, wherein a said wnt signal is detected by exposure of cells transfected with a reporter system containing TCF/LEF response elements in the promoter region of a reporter gene.

9. A method as claimed in claim 1 comprising performing on a compound a first screen, wherein a said wnt signal is detected in said conditioned medium by assaying for wnt mediated activation of bone formation by osteoblasts and a second screen in accordance with any one of claims 4 to 8, wherein a said wnt signal is detected in said conditioned medium by assaying for wnt mediated activation of LRP5 and or LRP6 signalling in a cell.
